(19) United States
(12) Patent Application Publication
(10) Pub. No.: US 2010/0255533 A1
(43) Pub. Date: Oct. 7, 2010

(54) Method for processing shark's cartilage marrow

(76) Inventor: Chun Yuan Chen, Taipei (TW)

Correspondence Address: Muncy, Geissler, Olds & Lowe, PLLC, 4000 Legato Road, Suite 310, FAIRFAX, VA, 22033, US

(21) Appl. No.: 12/149,929

(22) Filed: May 9, 2008

(51) Int. Cl. C12P 1/00

(52) U.S. Cl. 435/41

(57) ABSTRACT

A method for processing shark's cartilage marrow comprises steps of: (A). dissolving the shark's cartilage marrow by plant enzymes; (B). hydrolyzing the dissolved mixture obtained from step (A) with pure water and removing impurities from the hydrolyzed product by the specific gravity difference between the impurities and the hydrolyzed product; (C). homogenizing the product obtained from step (B) into an aqueous solution; (D). processing the aqueous solution with a precipitation procedure; and (E). removing excess water by chromatographic separation and obtaining shark's cartilage marrow extract (SDS; shark distillated for saccharide) via specific gravity difference.

### BACKGROUND OF THE INVENTION

1. Field of the Invention

The present invention relates to a method for processing cartilage marrow and, more particularly, to a method for processing shark's cartilage marrow to obtain highly purified shark's cartilage marrow extract (shark distillated for saccharide).

2. Description of the Prior Art

Recently, it has been found that shark's cartilage is helpful for medical treatments of some diseases. Especially, the anti-angiogenic activity of the shark's cartilage is the focus to be studied.

Conventionally, shark cartilage is usually only smashed or ground into powders and the powders obtained by this way is characterized by strong fishy smell and gray or black color that will be unattractive to consumers. Besides, effective components in shark cartilage are usually unable to be extracted therefrom by this method.

In order to solve problems mentioned above, the inventor of the present invention has proposed a method disclosed in his United States patent with the U.S. Pat. No. 6,069,436 and title “Method of obtaining extract of shark's cartilage”. As described in that patent's specification, this method mainly comprises steps of crushing, washing, purifying, eliminating fishy smell, and sterilizing by heating up. Consequently, pure white extract without fishy smell can be obtained through the method for processing shark cartilage disclosed in that patent.

According to the above patent, shark's cartilage is crushed by appropriative pressure and the marrow in the shark cartilage can be taken out when perichondrium is removed. Besides, the fishy smell can be eliminated by using appropriate enzymes. After disclosing the method of above patent, inventor had the motive to study and develop the present invention after hard research to provide a method for processing the shark's cartilage marrow that is obtained from the method in above patent and consequently an strikingly new method of extraction can be further obtained.

### SUMMARY OF THE INVENTION

An object of the present invention is to provide a method for processing shark's cartilage marrow to obtain highly purified shark's cartilage marrow extract (SDS; shark distillated for saccharide).

In order to achieve the above object, the present invention provides a method for processing shark's cartilage marrow, comprises steps of: (A). dissolving the shark's cartilage marrow by plant enzymes; (B). hydrolyzing the dissolved mixture obtained from step (A) with pure water and removing impurities from the hydrolyzed product by the specific gravity difference between the impurities and the hydrolyzed product; (C). homogenizing the product obtained from step (B) into an aqueous solution; (D). processing the aqueous solution with a precipitation procedure; and (E). removing excess water by chromatographic separation and obtaining shark's cartilage marrow extract (SDS) via specific gravity difference.

In practice, in step (A), the plant enzymes are preferably apple or pineapple enzymes. Besides, the shark's cartilage marrow is preferably processed in a closed container at a temperature between 45 and 48° C. for 4 to 6 hours.

The following detailed description, given by way of examples or embodiments, will best be understood in conjunction with the accompanying drawings.

### BRIEF DESCRIPTION OF THE DRAWINGS

FIG. 1 shows a flow chart of a method for processing shark's cartilage marrow of the present invention.

### DETAILED DESCRIPTION OF THE PREFERRED EMBODIMENTS

The present invention provides a method for processing shark's cartilage marrow in order to obtain highly purified shark's cartilage marrow extract (SDS; shark distillated for saccharide). The method comprises steps of enzyme dissolution, hydrolysis, homogenization, precipitation, and separation.

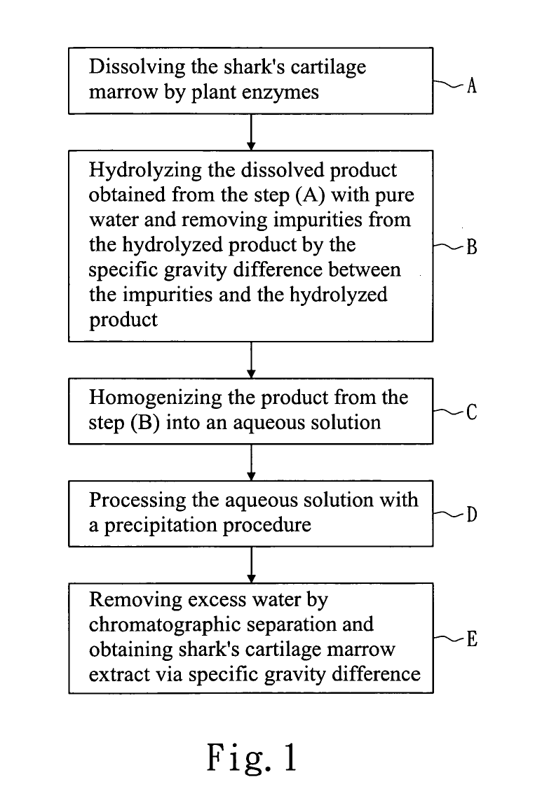

Please refer to FIG. 1, a flow chart of the method for processing shark's cartilage marrow according to the present invention is shown. The steps of the method are described in details in the following.

In step (A), plant enzymes are used to dissolve the shark's cartilage marrow. In a preferred embodiment, the plant enzymes used are apple enzymes or pineapple enzymes. Besides, the shark's cartilage marrow is dissolved at a temperature between 45 and 48° C. in a closed container for 4 to 6 hours.

As to step (B), the dissolved mixture obtained from step (A) is hydrolyzed with pure water and impurities are removed from the hydrolyzed product by the specific gravity difference between the impurities and the hydrolyzed product. Because the dissolved mixture obtained from step (A) still has some impurities, shark's cartilage marrow extract can be obtained via the specific gravity difference and the use of separating apparatus after the dissolved mixture is hydrolyzed with pure water.

In step (C), the product obtained from step (B) is completely homogenized into an aqueous solution. In a preferred embodiment, the shark's cartilage marrow extract obtained from step (B) are introduced into a centrifugal homogenizer to be homogenized. When the shark's cartilage marrow extract obtained form step (B) is completely homogenized into an aqueous solution, the water content of the aqueous solution is preferably 50%.

In step (D), the aqueous solution obtained from step (C) is processed with a precipitation procedure. In a preferred embodiment, precipitation procedure is to keep still the aqueous solution obtained from step (C) for 24 hours.

As to step (E), excess water is removed by chromatographic separation and shark's cartilage marrow extract (SDS) is obtained via specific gravity difference. In a preferred embodiment, excess water can be removed by using a chromatography and shark's cartilage marrow extract with high purity can be separated by means of chromatographic resins in the chromatography via specific gravity difference.

As disclosed in the above description and attached drawings, the present invention can provide a method for processing shark's cartilage marrow, comprising steps of enzyme dissolution, hydrolysis, homogenization, precipitation, and separation. Consequently, highly purified shark's cartilage marrow extract (SDS) can be obtained. It is new and can be put into industrial use.

Although the embodiments of the present invention have been described in detail, various modifications and variations may be made by those skilled in the art from the teachings disclosed hereinabove. Therefore, it should be understood that any modification and variation equivalent to the spirit of the present invention be regarded to fall into the scope defined by the appended claims.

What is claimed is:

1. A method for processing shark's cartilage marrow, comprises steps of:
A). dissolving the shark's cartilage marrow by plant enzymes;
B). hydrolyzing the dissolved mixture obtained from step (A) with pure water and removing impurities from the hydrolyzed product by the specific gravity difference between the impurities and the hydrolyzed product;
C). homogenizing the product obtained from step (B) into an aqueous solution;
D). processing the aqueous solution with a precipitation procedure; and
E). removing excess water by chromatographic separation and obtaining shark's cartilage marrow extract via specific gravity difference.

2. The method for processing shark's cartilage marrow as claimed in claim 1, wherein the shark's cartilage marrow is processed at a temperature between 45 and 48° C. in a closed container for 4 to 6 hours in step (A).

3. The method for processing shark's cartilage marrow as claimed in claim 1, wherein the plant enzymes used in step (A) are apple or pineapple enzymes.
